(19) United States
(12) Patent Application Publication
(10) Pub. No.: US 2024/0189151 A1
(43) Pub. Date: Jun. 13, 2024

(54) EYE SHIELD STRIP-AID METHOD AND DEVICES

(71) Applicant: Richard Bruno Muller, Marietta, GA (US)

(72) Inventor: Richard Bruno Muller, Marietta, GA (US)

(21) Appl. No.: 18/079,000

(22) Filed: Dec. 11, 2022

(51) Int. Cl. A61F 9/04

(52) U.S. Cl. CPC A61F 9/04; A61F 2250/0076; A61F 2250/0091

(57) ABSTRACT

The embodiments disclose an eye protector apparatus with an adhesive strip having an oval center body with an oval opening and a first tab protruding outward from the oval center body and a second tab protruding outward from the oval center body. Also, an eye shield is included that has an inner oval shape portion having at least two different sized holes to allow visibility and ventilation to an eye and configured to fit within the oval center body. The eye shield further includes first and second flexible tabs on opposites sides of the eye shield. The adhesive strip further includes a backside adhesive coating along a perimeter edge the oval opening configured to adhere the eye shield to the adhesive strip. Also, each of the first and second tabs have first adhesive portions configured to adhere respectively to the first and second flexible tabs of the eye shield and wherein the first and second tabs each have hypoallergenic lower adhesive portions configured to adhere respectively to the eyebrow of the user and the cheek of the user.

### BACKGROUND

The use of an eye shield, post-eye surgery, is required by all doctors who perform cataracts and most any other type of eye surgery globally. An eye shield is typically held in place over the patient eye using a medical-grade tape. The shield fits over either the left or the right eye. It is a cumbersome process/method for the patient to place the eye shield against one's face over the eye area and also place two or more strips of medical tape, in order to hold the shield in place when leaving the surgical procedure. The eye shield is also required to be placed over the patient's eye while sleeping.

### BRIEF DESCRIPTION OF THE DRAWINGS

FIG. 1 shows for illustrative purposes only an example of an overview of an eye shield strip-aid of one embodiment.

FIG. 2A shows for illustrative purposes only an example of strip-aid positioning for applying of one embodiment.

FIG. 2B shows for illustrative purposes only an example of a strip-aid applied to the eye shield of one embodiment.

FIG. 3A shows for illustrative purposes only an example of a strip-aid one-piece non-woven material exterior surface of one embodiment.

FIG. 3B shows for illustrative purposes only an example of strip-aid three-piece release liners of one embodiment.

FIG. 4A shows for illustrative purposes only an example of an eye shield front side with sight holes of one embodiment.

FIG. 4B shows for illustrative purposes only an example of an eye shield backside with sight holes of one embodiment.

FIG. 5 shows for illustrative purposes only an example of a post-op patient wearing the eye shield strip-aid of one embodiment.

FIG. 6A shows for illustrative purposes only an example of an eye shield flexible molded substrate extension of one embodiment.

FIG. 6B shows for illustrative purposes only an example of a strip-aid eyebrow and cheek area epidermis section of one embodiment.

FIG. 7A shows for illustrative purposes only an example of the smart strip-aid front view of one embodiment.

FIG. 7B shows for illustrative purposes only an example of the smart strip-aid back view of one embodiment.

FIG. 8 shows a block diagram of an overview of smart strip-aid sensors and mobile application of one embodiment.

FIG. 9A shows a block diagram of an overview of the smart strip-aid shower eye guard of one embodiment.

FIG. 9B shows a block diagram of an overview of a smart strip-aid shower eye guard attached to smart strip-aid of one embodiment.

FIG. 10A shows a block diagram of an overview of the smart strip-aid pad of one embodiment.

FIG. 10B shows a block diagram of an overview of the smart strip-aid pad position of one embodiment.

### DETAILED DESCRIPTION OF THE INVENTION

In a following description, reference is made to the accompanying drawings, which form a part hereof, and in which is shown by way of illustration a specific example in which the invention may be practiced. It is to be understood that other embodiments may be utilized, and structural changes may be made without departing from the scope of the present invention.

### General Overview

It should be noted that the descriptions that follow, for example, in terms of an eye shield strip-aid method and devices are described for illustrative purposes and the underlying system can apply to any number and multiple types of eye shield devices. In one embodiment of the present invention, the eye shield strip-aid method and devices can be configured using a plurality of non-woven materials for blending with skin colors and tones. The eye shield strip-aid method and devices can be configured to include sensors to detect bacteria and blood and can be configured to include a range of infection preventative treatments incorporated with the non-woven material and adhesives using the present invention.

Certain terminology is used for convenience only and is not to be taken as a limitation on the invention. For example, words such as “front side” and “backside” merely describe the configuration as shown in the figures. Indeed, the components may be oriented in two directions and the terminology therefore should be understood as encompassing such variations, unless specified otherwise.

The invention relates to a narrow part of the human body, mainly the eye, and how the invention can assist the consumer/patient to secure an eye shield more easily, over one's eye. The use of an eye shield, post-eye surgery, is required by all doctors who perform cataract surgery and most any other type of eye surgery globally. The drawings show one type of eye shield for illustrative purposes only that include the typical air flow sight holes incorporated in post-surgery eye shields. It is to be understood that the invention is useable on any eye shield and provides the same functions and elements on any eye shield.

The largest demographic of patients that require cataract eye surgery, along with all other types of eye surgery are people who are mature and generally over 60 years of age. As the global population continues to live longer in both the industrial and the emerging countries of the world, the volume of cataract surgery and other eye surgery will increase. This increase will occur more rapidly in areas of the world like Asia where there are tens of millions of people entering the middle class that will require eye surgery of all types.

The volume of these medical procedures will have a direct correlation to the countries' ecosystems and how those countries manage the world around them. Regions of the world that are plagued with dangerous ecosystems for humans, are especially more likely to experience higher volumes of eye-related medical problems requiring surgery.

In general, the invention includes an eye protector apparatus with an adhesive strip having an oval center body with an oval opening and a first tab protruding outward from the oval center body forming a first longitudinal terminus of the adhesive strip at a first end and a second tab protruding outward from the oval center body forming a second longitudinal terminus of the adhesive strip at a second end. Also, an eye shield is included that has an inner oval shape portion having at least two different sized holes to allow visibility and ventilation to an eye and configured to fit within the oval center body. The eye shield further includes first and second flexible tabs on opposites sides of the eye shield, wherein the first flexible tab is configured to rest on an eyebrow of a user and the second flexible tab is configured to rest on a cheek of the user. The adhesive strip further includes a backside adhesive coating along a perimeter edge the oval opening configured to adhere the eye shield to the adhesive strip. In addition, the first and second tabs each have first adhesive portions configured to adhere respectively to the first and second flexible tabs of the eye shield and wherein the first and second tabs each have hypoallergenic lower adhesive portions configured to adhere respectively to the eyebrow of the user and the cheek of the user.

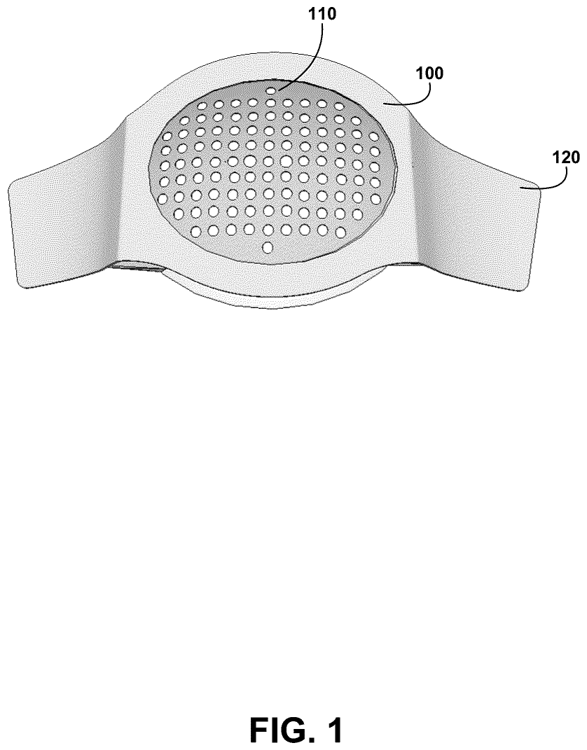

FIG. 1 shows for illustrative purposes only an example of an overview of the eye shield strip-aid of one embodiment. FIG. 1 shows an eye shield strip-aid 100 configured to attach an eye shield 110 covering a patient's eye after eye surgery. The eye shield strip-aid 100 includes a strip-aid with adhesive 120 to securely hold the eye shield 110 in position. The strip-aid with adhesive 120 includes an open section to leave eye shield 110 sight holes unblocked allowing the patient to see through the eye shield 110. In one embodiment, the eye shield can further include a removable clear waterproof film configured to temporarily cover the holes when the person is bathing or showing.

The strip-aid with adhesive 120 provides an improved method to secure the eye shield over one's eye after surgery, in place of just using a medical grade tape. The strip-aid with adhesive 120 firmly adheres to the eye shield 110 substrate to maintain the eye shield 110 position on the patient face. The strip-aid with adhesive 120 fits any eye shield over either the left or the right eye. The strip-aid with adhesive 120 also attaches the extended terminuses to an eyebrow area at one end and facial skin of the cheek at the opposite end of the strip-aid with adhesive 120. The adhesive is a medical-grade adhesive that includes anti-bacterial and antibiotic material to aid in preventing infection.

The strip-aid with adhesive 120 will eliminate a cumbersome process/method for one to place the eye shield against one's face over the eye area and also place two or more strips of medical tape, in order to hold the shield in place when leaving the surgical procedure. The eye shield is also required in a post-surgical eye operation to be placed over one's eye while sleeping, for at least seven (7) days.

FIG. 1 shows the eye shield contains an oval, inner egg shape portion of the substrate. The flexible molded part of the substrate is the portion of the product that rests on the epidermis of the eyebrow area at one end and facial skin of the cheek at the opposite end. This flexible molded hypoallergenic portion of the eye shield allows this shield to be comfortable and medically safe for all patients. The inner egg shape portion of the substrate contains two different size holes that allows for the flow of air to the eye along with the ability to see through the various holes.

In another embodiment, the invention may be made to appear the same size and shape but contain small square holes in the egg shape portion of the substrate. This embodiment allows the eye shield strip-aid 100 to be used to cover an industrial eye accident that may occur within a factory, plant, or on a job-site by a contract or full-time employee.

Accordingly, in yet another embodiment a reduction or enlargement of the size of the eye shield strip-aid 100 can be fitted onto the eye of a small child or a young adult. The eye shield strip-aid 100 sized for a small child can include character images familiar to small children to help parents and health care providers with the emotional impact of eye surgery performed on the small child. The characters may resemble animated characters seen on children's television shows and other images, for example, butterflies, koalas, and other images children enjoy.

In another embodiment, the eye shield strip-aid 100 generally has been invented for use by humans. The same combinations of these elements are applicable to pets of all kinds that may require a medically approved eye cover patch. Accordingly, we intend to cover all such modifications, additions, and equivalents as may be included within the spirit and scope of the invention of one embodiment.

### DETAILED DESCRIPTION

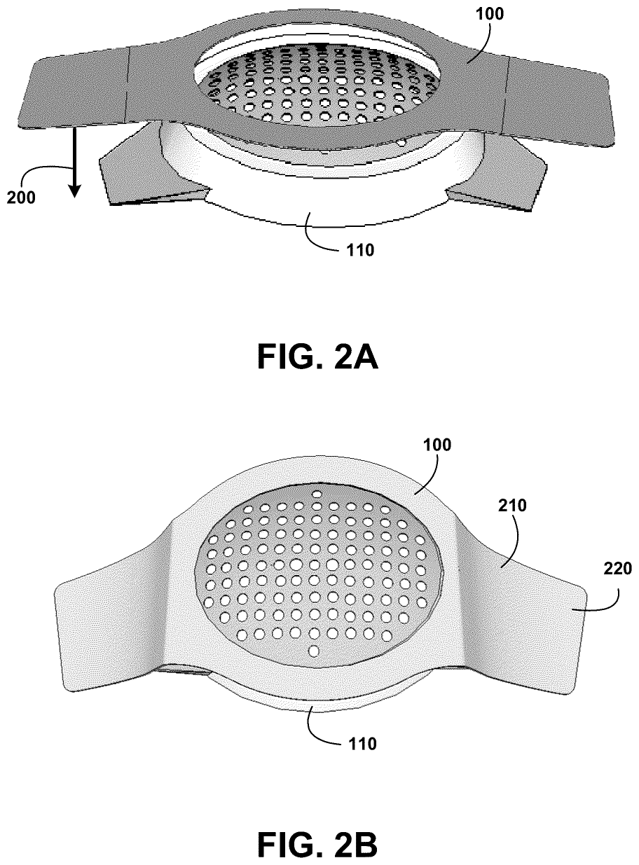

FIG. 2A shows for illustrative purposes only an example of the strip-aid positioning for applying of one embodiment. FIG. 2A shows an eye shield strip-aid 100 used to secure an eye shield 110 over a patient eye post-eye surgery. The patient can lower the strip-aid to eye shield 200 to secure the eye shield strip-aid 100 to the eye shield substrate prior to placing the eye shield over the eye of one embodiment.

### Strip-Aid Applied to Eye Shield:

FIG. 2B shows for illustrative purposes only an example of a strip-aid applied to the eye shield of one embodiment. FIG. 2B shows the eye shield strip-aid 100 attached to the eye shield 110. The eye shield strip-aid 100 adhesive provides secure adherence to the eye shield substrate side wall and extended terminuses 210. The eye shield strip-aid 100 extends beyond the extended terminuses to provide adherence to user skin 220 of one embodiment.

### Strip-Aid One-Piece Non-Woven Material Exterior Surface:

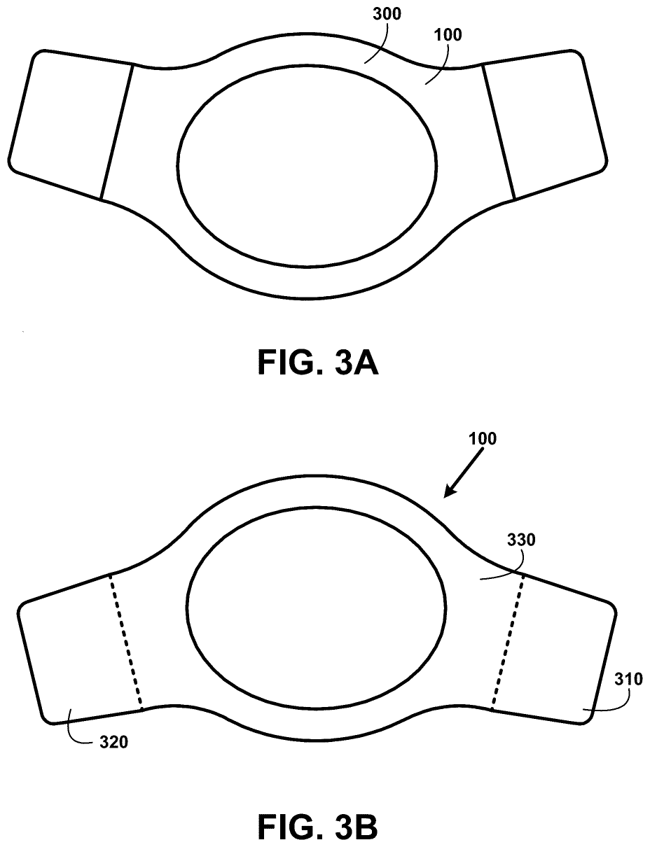

FIG. 3A shows for illustrative purposes only an example of a strip-aid one-piece non-woven material exterior surface of one embodiment. FIG. 3A shows the eye shield strip-aid 100 with a strip-aid one-piece non-woven material exterior surface 300. The strip-aid one-piece non-woven material exterior surface 300 when attached to the eye shield 110 of FIG. 1 eliminates the patient from having to place two or more strips of medical tape while keeping the eye shield 110 of FIG. 1 properly positioned against one's face over the eye area of one embodiment.

### Strip-Aid Three-Piece Release Liners:

FIG. 3B shows for illustrative purposes only an example of strip-aid three-piece release liners of one embodiment. FIG. 3B shows a backside view of the eye shield strip-aid 100. The backside of the eye shield strip-aid 100 shows three release liners that are covering the adhesive until the patient is ready to apply the eye shield strip-aid 100 to the eye shield 110 of FIG. 1. An eye shield first release liner 330 is easily peeled-off first to attach the eye shield strip-aid 100 to the substrate of the eye shield 110 of FIG. 1. A second terminus release liner 310 is easily peeled-off to expose the adhesive. A third terminus release liner 320 is easily peeled-off to expose the adhesive. The second terminus release liner 310 and third terminus release liner 320 are then removed to allow adherence to the eye shield 110 of FIG. 1 over the affected eye to the patient face eyebrow area at one end and facial skin of the cheek at the opposite end. This final step will complete the task allowing the patient to fit the eye shield over their eye without the help of another person. The adhesive strip having an oval center body with an oval opening and a first tab protruding outward from the oval center body forming a first longitudinal terminus of the adhesive strip at a first end and a second tab protruding outward from the oval center body forming a second longitudinal terminus of the adhesive strip at a second end of one embodiment.

Eye Shield Front Side with Sight Holes:

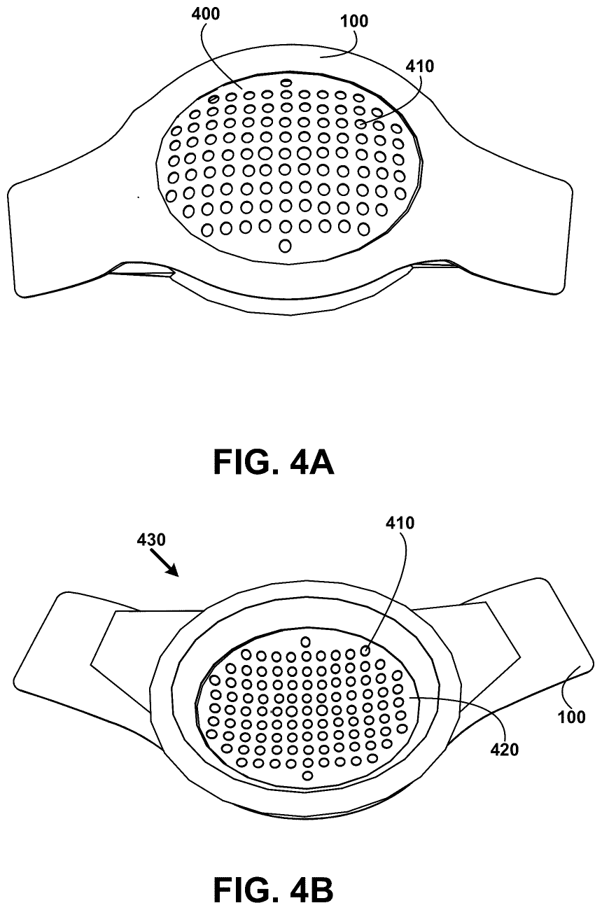

FIG. 4A shows for illustrative purposes only an example of an eye shield front side with sight holes of one embodiment. FIG. 4A shows the eye shield 110 of FIG. 1 in a view of an eye shield substrate front side 400. The front side view shows the air flow sight holes 410. These holes allow a patient to see through the holes while also allowing airflow to enter through the same small holes. The oval-shaped opening in the eye shield strip-aid 100 provides unobstructed sight through the air flow sight holes 410. The oval-shaped opening in the eye shield strip-aid 100 allows maintaining airflow to the eye while wearing the eye shield 110 of FIG. 1. The substrate contains two different size holes that allow for the flow of air to the eye along with the ability to see through the various holes of one embodiment.

Eye Shield Backside with Sight Holes:

FIG. 4B shows for illustrative purposes only an example of an eye shield backside with sight holes of one embodiment. FIG. 4B shows a backside view 430 of the eye shield 110 of FIG. 1. The eye shield substrate 420 back side view 430 illustrates the air flow sight holes 410 are open through the eye shield substrate 420. The oval-shaped opening in the eye shield strip-aid 100 allows an unobstructed clear view for the patient through the air flow sight holes 410. The first and second tabs each having a first adhesive portions configured to adhere respectively to the first and second flexible tabs of the eye shield and wherein the first and second tabs each having hypoallergenic lower adhesive portions configured to adhere respectively to the eyebrow of the user and the check of the user of one embodiment.

### Post-Op Patient Wearing the Eye Shield Strip-Aid:

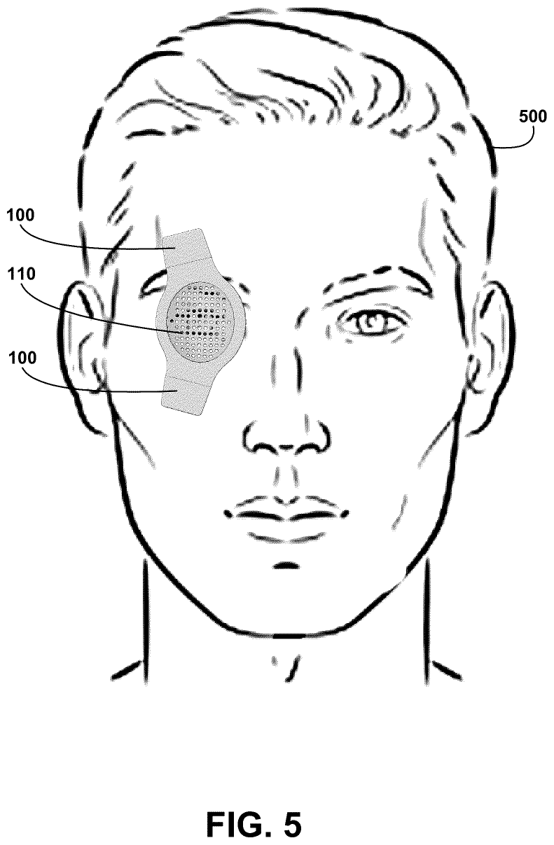

FIG. 5 shows for illustrative purposes only an example of a post-op patient wearing the eye shield strip-aid of one embodiment. FIG. 5 shows an eye surgery post-op patient 500 wearing the eye shield strip-aid 100 keeping the eye shield 110 properly positioned over the affected eye. The eye shield strip-aid 100 easily adheres to the eye shield 110 to fit over either the left or the right eye. The eye shield strip-aid 100 includes easy instructions on how to remove all three (3) release liners from off the backside of the eye shield strip-aid 100. For example the instructions may include the following steps including Step one: Remove center adhesive strip release liner off the eye shield strip-aid and place on eye shield. The instructions recommend placing the eye shield on a clean table/counter for this task. Step two: Remove bottom adhesive strip release liner off the eye shield strip-aid and secure to ones cheek area. User must make sure the placement of the eye shield is placed over ones post-op eye fully. Step three: Remove top adhesive strip release liner off the eye shield strip-aid and placed securely onto ones forehead. The eye shield further includes first and second flexible tabs on opposite sides of the eye shield, wherein the first flexible tab is configured to rest on an eyebrow of a user and the second flexible tab is configured to rest on a cheek of the user of one embodiment.

The instructions are short and easy to follow relieving the patient of the cumbersome process to place the eye shield against one's face over the eye area. The eye shield strip-aid 100 replaces placing two or more strips of medical tape to hold the eye shield in place. This task is cumbersome at best even for patients that have good dexterity when using their hands. The eye shield strip-aid 100 is for use by homeowners, hospitals caregivers, industrial and manufacturing facilities, government agencies, (i.e., military persons), public, private, tech schools, colleges, and universities worldwide in their sports programs and in their Sick/Health Rooms of one embodiment.

### Eye Shield Flexible Molded Substrate Extension:

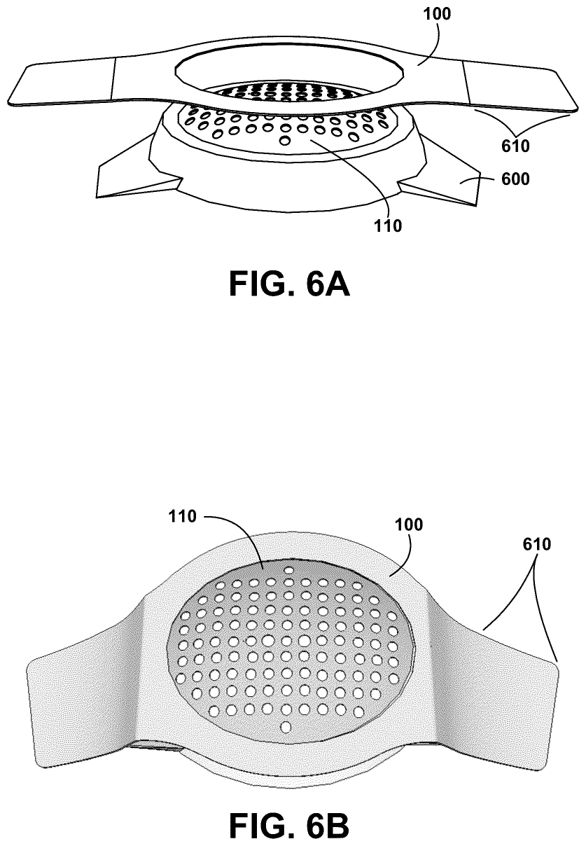

FIG. 6A shows for illustrative purposes only an example of an eye shield flexible molded substrate extension of one embodiment. FIG. 6A shows the eye shield strip-aid 100 positioned over the eye shield 110 and two flexible molded substrate extension 600 projections. The eye shield strip-aid 100 is made with a non-woven material that is of medical-grade quality. The non-woven material includes a medical grade flexible non-woven material that is flesh colored. In another embodiment the eye shield strip-aid 100 is made using a medical grade flexible fabric that is flesh colored. The flesh coloring is provided in various colors and tones to match a majority of skin tones covering a diverse population.

The medical grade flexible non-woven material easily conforms to the shape of the strip-aid eyebrow area at one end and facial skin of the cheek at the opposite end epidermis section 610 on both sides. Both the flexible non-woven material and the medical grade flexible fabric are hypoallergenic, strong, and tear-resistant but not ridged, allowing the eye shield strip-aid 100 to fit precisely over the top side of the eye shield. The eye shield strip-aid 100 fits perfectly leaving the eye shield air flow and sight holes open while securing the eye shield with the least amount of trouble by the patient. The eye shield has an inner oval shape portion having at least two different sized holes to allow visibility and ventilation to an eye and configured to fit within the oval center body of one embodiment.

### Strip-Aid Eyebrow and Cheek Area Epidermis Section:

FIG. 6B shows for illustrative purposes only an example of a strip-aid eyebrow and cheek area epidermis section of one embodiment. FIG. 6B shows the eye shield strip-aid 100 adhered to the eye shield 110. The strip-aid eyebrow and cheek area epidermis section 610 has the flexibility to adhere to the eye shield 110 substrate contours. The strip-aid eyebrow and cheek area epidermis section 610 flexibilities allows the patient to press the extended terminuses against the eyebrow and cheek area for full adherence to the contours of the facial eyebrow and cheek shapes of one embodiment. The eye shield can further include a third flexible section in between the first and second sections, wherein the third flexible section is configured to rest near a temple of a user. The adhesive strip further includes a backside adhesive coating along a perimeter edge the oval opening configured to adhere the adhesive strip to the eye shield of one embodiment.

### Smart Strip-Aid Front View:

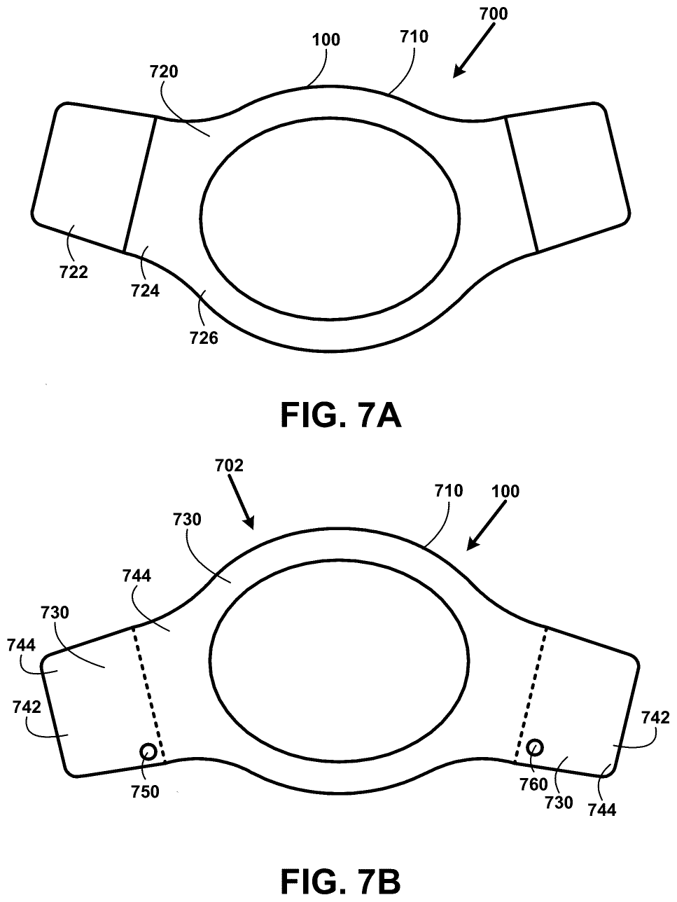

FIG. 7A shows for illustrative purposes only an example of the smart strip-aid front view of one embodiment. FIG. 7A shows a smart strip-aid 100 in a front view 700. The smart strip-aid 100 is made in one embodiment with a non-woven material 710 that blends into the skin color and tones 720 of the patient. The skin color and tones are varying to match a majority of a diverse population. The non-woven materials 710 is treated with anti-bacterial 722, sunblock 724, and antibiotic 726 materials to aid in infection prevention of one embodiment.

### Smart Strip-Aid Back View:

FIG. 7B shows for illustrative purposes only an example of the smart strip-aid back view of one embodiment. FIG. 7B shows the smart strip-aid 100 in a back view 702. A medical grade adhesive 730 on the backside of the non-woven material 710 is easily removable 740, sterile 742 (such as a material with a sterile barrier), and hypoallergenic 744. In one embodiment, the smart strip-aid 100 can easily be removed using an alcohol pad to release the adhesive. In one embodiment, the smart strip-aid 100 includes a sensor to detect blood and send an alert to the user's smartphone 750. In another embodiment, the smart strip-aid 100 includes a sensor to detect bacteria and send an alert to the user's smartphone 760. The smart strip-aid 100 aids both the patient and eye surgeon in post-operative monitoring of the patient's condition and infection prevention of one embodiment.

### Smart Strip-Aid Sensors and Mobile Application:

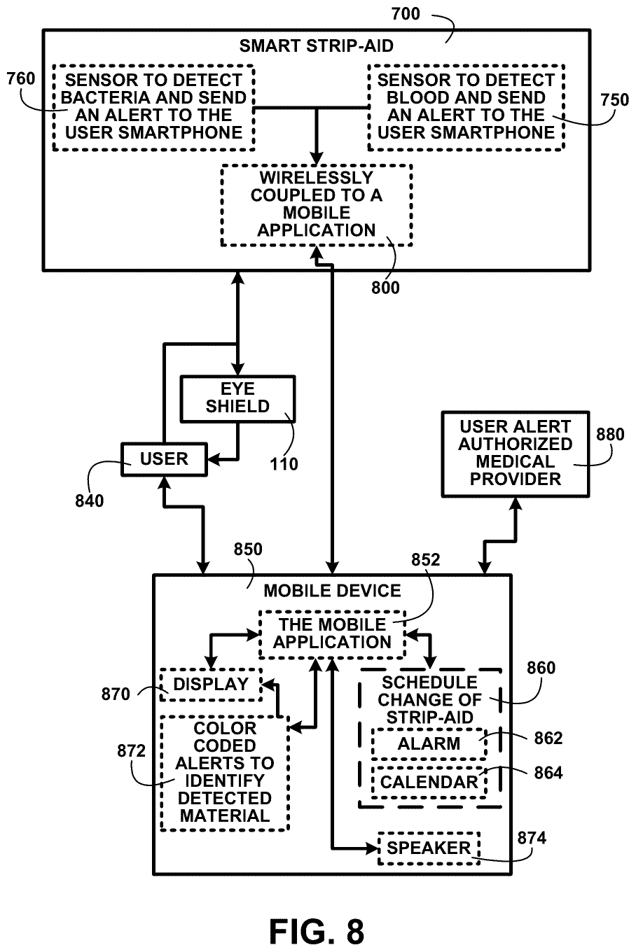

FIG. 8 shows a block diagram of an overview of smart strip-aid sensors and mobile application of one embodiment. FIG. 8 shows the smart strip-aid 700 with the sensor to detect blood and send an alert to the user's smartphone 750 and the sensor to detect bacteria and send an alert to the user's smartphone 760. The sensors are wirelessly coupled to a mobile application 800. The sensors coupled to the smart strip-aid are located in proximity and attached to the eye shield 110 placed on the user 840. A user 840 mobile device 850 with an installation of a smart strip-aid 700 mobile application 852 in one embodiment receives alerts of, for example, the presence of blood and/or bacteria. The user alert authorized medical provider 880 automatically receives the alerts and can advise the user on how to proceed.

The user can schedule a change of strip-aid 860 per medical instructions. The schedule change of strip-aid 860 is displayed by the mobile application 852 on the mobile device 850 for example on a calendar 864. A mobile device 850 speaker 874 can sound an audible alarm 862 and, for example, a verbal message with text-to-voice capability. An alarm 862 can also display 870 color-coded alerts to identify detected material 872 including blood and bacteria of one embodiment.

### Smart Strip-Aid Shower Eye Guard:

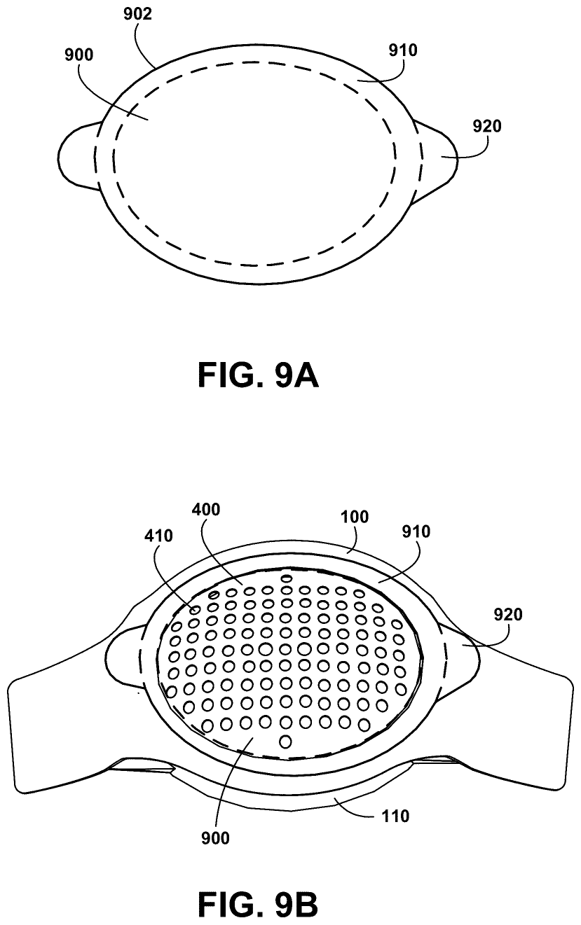

FIG. 9A shows for illustrative purposes only an example of a smart strip-aid shower eye guard of one embodiment. FIG. 9A shows a waterproof transparent sight hole covering 900. The waterproof transparent sight hole covering 900 is a smart strip-aid shower eye guard 902 to protect the post-op eye from contamination while the patient is showering. The smart strip-aid shower eye guard 902 includes a waterproof transparent backside adhesive strip 910. The waterproof transparent backside adhesive strip 910 is a coating of low-adherent adhesive that can be securely attached for 24 hours but is easily removed without dislodging the smart strip-aid 100 of FIG. 1 from the eye shield 110 of FIG. 1. The patient can pull either waterproof transparent peel-off tab 920 to remove the smart strip-aid shower eye guard 902 of one embodiment.

### Smart Strip-Aid Shower Eye Guard Attached to Smart Strip-Aid:

FIG. 9B shows for illustrative purposes only an example of a smart strip-aid shower eye guard attached to a smart strip-aid of one embodiment. FIG. 9B shows the eye shield substrate front side 400 with the eye shield strip-aid 100 attached to the eye shield 110. The waterproof transparent sight hole covering 900 is clear and allows a patient continuous vision through the air flow sight holes 410. A waterproof transparent sight hole covering backside adhesive strip 910 is used to temporarily attach the waterproof transparent sight hole covering 900 over the air flow sight holes 410 while a patient showers.

The waterproof transparent sight hole covering 900 prevents shower water from passing through the air flow sight holes 410 and potentially contaminating the recent eye surgery area. The patient after showering can pull either of the two waterproof transparent peel-off tabs 920 to remove the waterproof transparent sight hole covering 900. The waterproof transparent sight hole covering backside adhesive strip 910 has a sensitive skin adhesive that is easily peeled-off without pulling on the eye shield strip-aid 100 and dislodging it from the eye shield 110 of one embodiment.

### Smart Strip-Aid Pad:

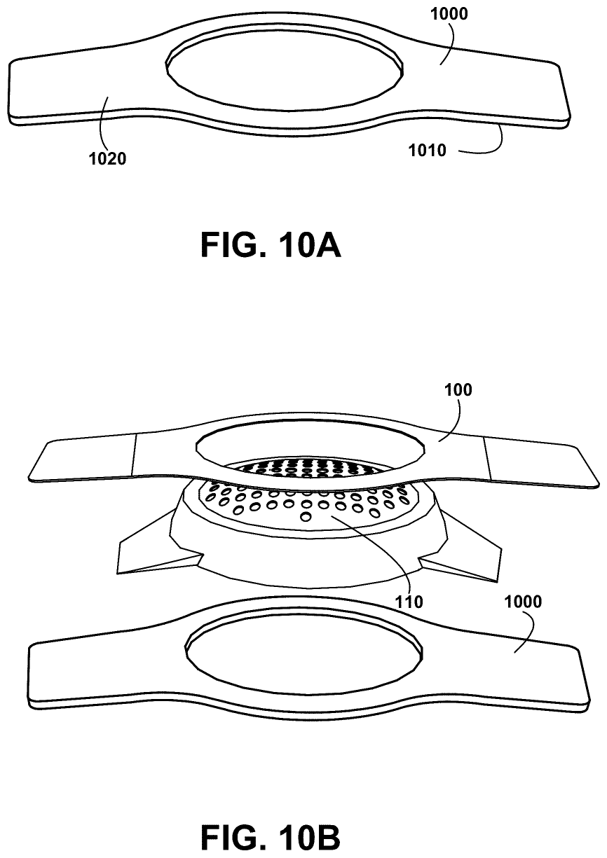

FIG. 10A shows for illustrative purposes only an example of a smart strip-aid pad of one embodiment. FIG. 10A shows a smart strip-aid sterile pad 1000. The smart strip-aid sterile pad 1000 on one side has a coating of a low adherent adhesive for easy removal from tender eye area skin 1010. The smart strip-aid sterile pad 1000 is in one embodiment the pad is medical grade sterile conforming gauze with at least 3 plies impregnated with an antibiotic and antiseptic treatment 1020 to prevent infection. In another embodiment, the pad can be a nonwoven gauze sponge impregnated with an antibiotic and antiseptic treatment 1020. The smart strip-aid sterile pad 1000 provides a soft cushion against the patient skin from the substrate of the eye shield 110 of FIG. 1. The adhesive backing helps prevent external contamination from entering the surgery area of one embodiment.

### Smart Strip-Aid Pad Position:

FIG. 10B shows for illustrative purposes only an example of a smart strip-aid pad position of one embodiment. FIG. 10B shows the smart strip-aid sterile pad 1000 against the patient skin to cushion the eye shield 110 attached by the eye shield strip-aid 100. The smart strip-aid sterile pad 1000 low adherent adhesive allows the patient to remove the pad without sticking to the tender eye area skin. The low adherent adhesive adherence will keep the pad in place even when wet until it is painlessly removed without sticking to the sensitive eye area skin. The impregnated antibiotic and antiseptic treatment of the smart strip-aid sterile pad 1000 protects against infection of one embodiment.

The foregoing has described the principles, embodiments, and modes of operation of the present invention. However, the invention should not be construed as being limited to the particular embodiments discussed. The above-described embodiments should be regarded as illustrative rather than restrictive, and it should be appreciated that variations may be made in those embodiments by workers skilled in the art without departing from the scope of the present invention as defined by the following claims.

1. An eye protector apparatus for an eye of a user, comprising:
an adhesive strip having an oval center body with an oval opening and a first tab protruding outward from the oval center body forming a first longitudinal terminus of the adhesive strip at a first end and a second tab protruding outward from the oval center body forming a second longitudinal terminus of the adhesive strip at a second end;
an eye shield located within the oval center body and having an inner oval shape portion having at least two different sized holes to allow visibility and ventilation to the eye of the user;
first and second flexible extensions on opposites sides of the eye shield, wherein the first flexible extension is configured to rest on an eyebrow of a user and the second flexible extension is configured to rest on a cheek of the user;
wherein the adhesive strip further includes a backside adhesive coating located along a perimeter edge of the oval opening and configured to adhere the eye shield to the adhesive strip; and
wherein each of the first and second tabs have first adhesive portions configured to adhere respectively to the first and second flexible tabs of the eye shield and wherein each of the first and second tabs have hypoallergenic lower adhesive portions configured to adhere respectively to the eyebrow of the user and the cheek of the user;
at least one sensor coupled to the eye shield and located in close proximity to the eye of the user and configured to detect bacteria in surrounding areas and in close proximity to the eye of the user; and
an eye guard mobile application with a graphical user interface and an alert processor operating on a mobile device of the user, wherein the alert processor is wirelessly coupled to the at least one sensor and wherein the eye guard mobile application is configured to display alerts on the graphical user interface when bacteria is detected by the at least one sensor.

2. The eye protector apparatus for an eye of a user of claim 1, further comprising a speaker of the user's mobile device configured to broadcast an audible alarm along with the display alerts on the graphical user interface.

3. The eye protector apparatus for an eye of a user of claim 1, wherein the adhesive strip includes a sterile pad made of medical grade sterile conforming gauze with at least three plies impregnated with an antibiotic and antiseptic treatment to prevent infection.

4. The eye protector apparatus for an eye of a user of claim 1, further comprising a shower eye guard having a waterproof transparent covering removably coupled to the eye shield configured to cover a sight area to protect the eye from fluids during bathing.

5. The eye protector apparatus for an eye of a user of claim 1, further comprising a speaker coupled to the mobile device of the user configured to broadcast a verbal sensor detection alert using an alert processor text to voice to vocalize the displayed alerts on the graphical user interface of the user's mobile device.

6. The eye protector apparatus for an eye of a user of claim 1, wherein the at least one sensor is further configured to detect blood.

7. The eye protector apparatus for an eye of a user of claim 1, wherein the mobile application is configured to transmit alerts to an authorized medical provider to advise the user on how to proceed.

8. An apparatus for protecting an eye after eye surgery, comprising:
an adhesive strip having an oval center body with an oval opening and a first tab protruding outward from the oval center body forming a first longitudinal terminus of the adhesive strip at a first end and a second tab protruding outward from the oval center body forming a second longitudinal terminus of the adhesive strip at a second end;
an eye shield located within the oval center body and having an inner oval shape portion having plural holes to allow visibility and ventilation to the eye of the user;
first[,] and second and third flexible extensions on opposite[s] sides of the eye shield, wherein the first flexible extension is configured to rest near on an eyebrow of a user[,] and the second flexible extension is configured to rest near on a cheek of the user;
wherein the adhesive strip further includes a backside adhesive coating located along a perimeter edge of the oval opening and configured to adhere the eye shield to the adhesive strip;
wherein each of the first and second tabs have first adhesive portions configured to adhere respectively to the first and second flexible tabs of the eye shield and wherein each of the first and second tabs have hypoallergenic lower adhesive portions configured to adhere respectively to the eyebrow of the user and the cheek of the user;
at least one sensor coupled to the eye shield and located in close proximity to the eye of the user and configured to detect bacteria in surrounding areas and in close proximity to the eye of the user; and
an eye guard mobile application with a graphical user interface and an alert processor operating on a mobile device of the user, wherein the alert processor is wirelessly coupled to the at least one sensor and wherein the eye guard mobile application is configured to display alerts on the graphical user interface when bacteria is detected by the at least one sensor.

9. The apparatus for protecting an eye after eye surgery of claim 8, wherein the mobile application includes a calendar processor to allow the user to schedule a change of strip-aid per medical instructions.

10. The apparatus for protecting an eye after eye surgery of claim 8, wherein the adhesive strip includes a sterile pad made of medical grade sterile conforming gauze with at least three plies impregnated with an antibiotic and antiseptic treatment to prevent infection.

11. The apparatus for protecting an eye after eye surgery of claim 8, further comprising a speaker integrated in the mobile device and configured to broadcast the alerts with an audible alarm and a text to voice conversion of the alerts.

12. The apparatus for protecting an eye after eye surgery of claim 8, wherein the mobile application is further configured to transmit the alerts to an authorized medical provider to advise the user on how to proceed.

13. The apparatus for protecting an eye after eye surgery of claim 8, wherein the at least one sensor is further configured to detect blood.

14. A method, comprising:
providing an adhesive strip having an oval center body with an oval opening and a first tab protruding outward from the oval center body forming a first longitudinal terminus of the adhesive strip at a first end and a second tab protruding outward from the oval center body forming a second longitudinal terminus of the adhesive strip at a second end;
providing an eye shield located within the oval center body and having an inner oval shape portion having at least two different sized holes to allow visibility and ventilation to the eye of the user;
resting first and second flexible extensions on opposites sides of the eye shield, wherein the first flexible extension is configured to rest on an eyebrow of a user[;] and the second flexible extension is configured to rest on a cheek of the user;
providing the adhesive strip further includes a backside adhesive coating located along a perimeter edge of the oval opening and configured to adhere the eye shield to the adhesive strip;
adhering each of the first and second tabs that have first adhesive portions configured to adhere respectively to the first and second flexible tabs of the eye shield[;] and wherein each of the first and second tabs that have hypoallergenic lower adhesive portions configured to adhere respectively to the eyebrow of the user and the cheek of the user;
providing at least one sensor coupled to the eye shield in close proximity to the eye of the user and configured to detect bacteria in surrounding areas and in close proximity to the eye of the user; and
operating an eye guard mobile application with a graphical user interface and an alert processor on a mobile device of the user, wherein the alert processor is wirelessly coupled to the at least one sensor and wherein the eye guard mobile application is configured to display alerts on the graphical user interface when bacteria is detected by the at least one sensor.

15. The method of claim 14, further comprising providing the adhesive strip and eye shield as a medically safe hypoallergenic material.

16. The method of claim 14, further comprising providing the adhesive strip as a sterile pad made of medical grade sterile conforming gauze with at least three plies impregnated with an antibiotic and antiseptic treatment to prevent infection.

17. The method of claim 14, further comprising using a speaker of the mobile device to broadcast the alerts with audible alarms and as verbal alerts that convert text to voice.

18. The method of claim 14, further comprising providing a third flexible extension on the eye shield that is located between the first and second extensions and resting the third flexible extension near a temple of the user.

19. The method of claim 14, further comprising detecting blood using the at least one sensor.

20. The method of claim 14, operating the mobile application to transmit the alerts to a user authorized medical provider to advise the user on how to proceed.
